(19) United States
(12) Patent Application Publication
(10) Pub. No.: US 2008/0269510 A1
(43) Pub. Date: Oct. 30, 2008

(54) New alpha-glucosidase inhibitors and antibacterial compounds from Myrtus communis L.

(75) Inventors: Attaur Rahman, Karachi (PK); Mohammad Iqbal Choudhary, Karachi (PK); Farzana Shaheen, Karachi (PK); Manzoor Ahmad, Karachi (PK); Shamsun Nahar Khan, Dhaka (BD); Shazia Anjum, Karachi (PK)

Correspondence Address: SARFARAZ K. NIAZI, 20 RIVERSIDE DRIVE, DEERFIELD, IL, 60015, US

(73) Assignee: INTERNATIONAL INSTITUTE OF CHEMICAL SCIENCES, Karachi (PK)

(21) Appl. No.: 11/306,345

(22) Filed: Dec. 23, 2005

(51) Int. Cl. C07D 493/04; C07D 311/80

(52) U.S. Cl. 549/382; 549/391

(57) ABSTRACT

Three new acylphloroglucinols, myrtucommulone-D (Compound 1), myrtucommulone-E (Compound 2), myrtucommulone-C (Compound 3), and a known acyphloroglucinol myrtucommulone B (Compound 4) were isolated from a methanolic extract of Myrtus communis L. The structures of compounds 1, 2 and 4 were also unambiguously determined by single X-ray diffraction analysis. The compounds 1-4 were found to be more potent α-glucosidase inhibitors than the clinically used standards, acarbose and deoxynojirimycin. The compound 3 exhibited the highest activity among all the acylphloroglucinols, with an IC₅₀=35.4±1.15 μM. The compounds 1 and 2 also exhibited strong antibacterial activities.

### BACKGROUND

Myrtus communis L. (Myrtaceae) commonly called as Myrtle (English) is an evergreen shrub and is widely distributed in Mediterranean region. Myrtle has been used as a folk medicine in several remedies (Feist C., Frank L., Appendino G., and Werz O., The Journal of Pharmacology and Experimental Therapeutics, 2005, 375(1), 389-396; Sepici A., Gurbuz I., Cevik C., and Yesilada E., Journal of Ethnopharmacology, 2004, 93, 311-318; Watt G. Dictionary of the Economic Products of India, 1972, II, p. 316, Cosmo Publication, Delhi-6, India; Mulas M., Spano D., Biscaro S., Parpinello L., Ind. Bevande, 2000, 29, 494-498; chem. Abstr. 2000, 734, 265512; Winter A. G., Willeke L., Naturewissenschaften, 1951, 38, 262-264.)

The characteristic constituents of this plant include monoterpenoids, flavonoids, triterpenoids, and phloroglucinol-type compounds (Rosa A., Deiana M., Casu V., Corona G., Appendino G., Bianchi F., Ballero M., and Dessi M. A., Free Radic. Res., 2003, 37(9), 1013-1019; Diaz A. M., Abeger A., Fitoterapia, 1997, 58, 167-174; Rotstein A., Lifshitz A., Kash man Y., Isr. Antimicrob. Agents Chemotherapy. 1974, 6, 539-542; Kashman Y., Rotstein A., and Lifshitz A., Tetrahedron, 1974, 30, 991-997; Appendino G., Bianchi F., Minassi A., Sterner O., Ballero M., and Gibbons S., J. Nat. Prod., 2002, 65, 334-338.)

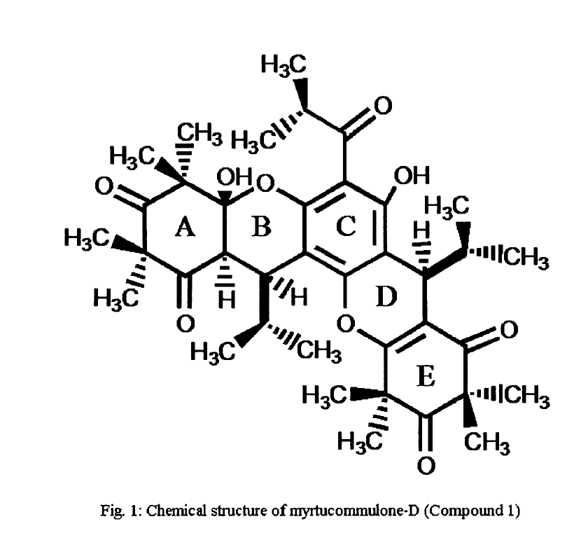

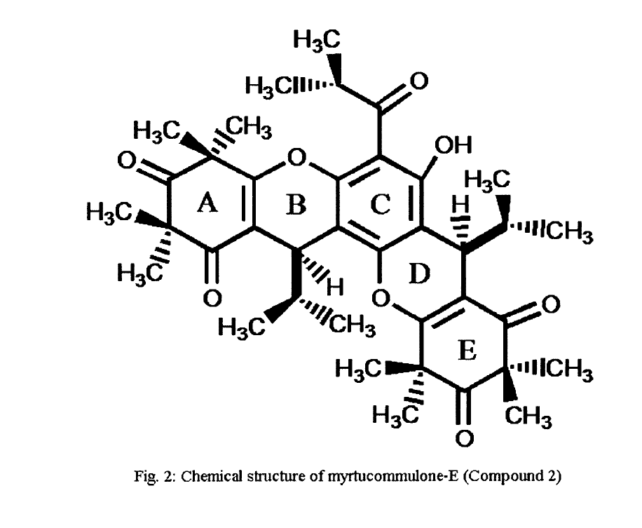

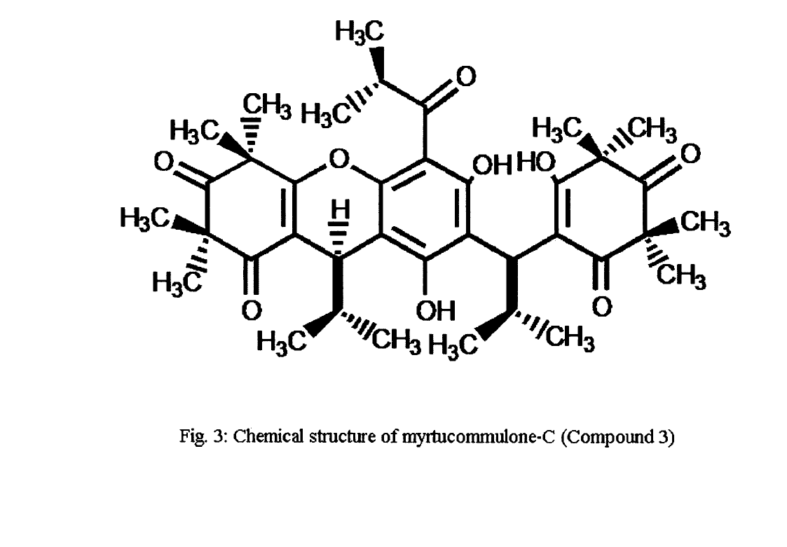

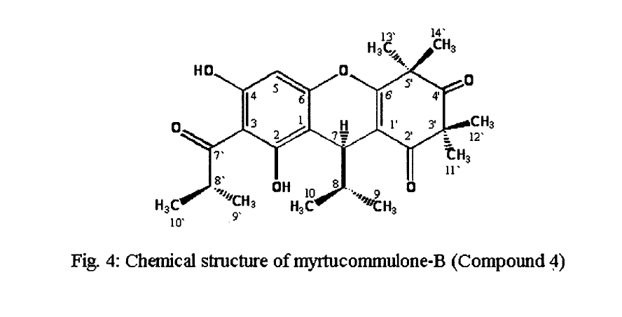

Three new acylphloroglucinols, myrtucommulone-D (Compound 1) (FIG. 1), myrtucommulone-E (Compound 2) (FIG. 2), myrtucommulone-C (Compound 3) (FIG. 3), along with a known compound myrtucommulone-B (4) (FIG. 4), seven triterpenes (compounds 7 and compounds 10-15) and two flavonoids (compounds 5 and compound 8) and 2,5-dihydroxy-4-methoxybenzophenone (cearoin), have been isolated from Myrtus communis L.

FIG. 1: Chemical Structure of Myrtucommulone-D (Compound 1)

FIG. 2: Chemical Structure of Myrtucommulone-E (Compound 2)

FIG. 3: Chemical Structure of Myrtucommulone-C (Compound 3)

FIG. 4: Chemical Structure of Myrtucommulone-B Compound 4)

The structures of compounds 1, 2 and 4 were also unambiguously determined by single X-ray diffraction analysis. The compounds 1-4 were found to be more potent α-glucosidase inhibitors than the clinically used standards, acarbose and deoxynojirimycin. The compound 3 exhibited the highest activity among all the acylphloroglucinols, with an IC₅₀=35.4±1.15 μM. The compounds 1, 2, 7, 9, and 11-15 have also exhibited antibacterial activities.

Several studies have revealed the strong antibacterial, anti-inflammatory, anti-hyperglycemic, antioxidant activities in the various extracts of this plant. (Feist C., Frank L., Appendino G., and Werz O., The Journal of Pharmacology and Experimental Therapeutics, 2005, 375(1), 389-396; Hayder N., Abdelwahed A., Kilani S., Ammar R. B., Mahmoud A., Ghedira K., and Chekir-Ghedira L., Mutat. Res., 2004, 564(1), 89-95; Romani A., Coinu R., Carta S., Pinelli P., Galardi C., Vincieri F. F., and Franconi F., Free Radic. Res., 2004, 38(1), 97-103; Bonjar G. H., Fitoterapia, 2004, 75(2), 231-235; Rosa A., Deiana M., Casu V., Corona G., Appendino G., Bianchi F., Ballero M., and Dessi M. A., Free Radic. Res., 2003, 37(9), 1013-1019.)

The effect of extracts from leaves of Myrtus communis L., on the SOS response induced by aflatoxin B1 (AFB1) and nifuroxazide using a bacterial assay system, i.e. the SOS chromotest with Escherichia coli PQ37 shows that the aqueous extract, the total flavonoids oligomer fraction (TOF), hexane, chloroform, ethyl acetate and methanol extracts and essential oil obtained from Myrtus communis L., significantly decrease the SOS response induced by AFB1 (10 mcg/assay) and nifuroxazide (20 mcg/assay). [SOS response is defined as the repair systems (recA; uvr) induced by the presence of single-stranded DNA that usually occurs from postreplicative gaps caused by various types of DNA damage. The RecA protein, stimulated by single-stranded DNA, is involved in the inactivation of the LexA repressor thereby inducing the response.] Ethyl acetate and methanol extracts show the strongest inhibition of the induction of the SOS response by the indirectly genotoxic AFB1. The methanol and aqueous extracts exhibit the highest level of protection towards the SOS-induced response by the directly genotoxic nifuroxazide. In addition to anti-genotoxic activity, the aqueous extract, the TOF, and the ethyl acetate and methanol extracts shows an important free-radical scavenging activity towards the 1,1-diphenyl-2-picrylhydrazyl (DPPH) radical. These observations suggest possible role of these extracts as additives in chemoprevention studies. (Hayder N, Abdelwahed A, Kilani S, Ammar R B, Mahmoud A, Ghedira K, Chekir-Ghedira L. Anti-genotoxic and free-radical scavenging activities of extracts from (Tunisian) Myrtus communis L. Mutat Res. 2004 Nov. 14; 564(1):89-95.)

Myrtus communis L., leaves as well as the volatile oil obtained from the leaves are used to lower the blood glucose level in type-2 diabetic patients in Turkish folk medicine. However, controlled studies show that oil did not show any effect in normoglycaemic rabbits either in single or multiple dose administrations, but a good hypoglycemic activity is observed 4 h after the administration to diabetic animals at 50 mg/kg in alloxan-diabetic rabbits. To investigate the effect of oil on repeated administration in both normal and diabetic rabbits, it was administered in 50 and 100 mg/kg doses once a day for one week. The oil significantly lowered blood glucose by 51% in alloxan-diabetic rabbits on the fourth hour and the following days at a dose of 50 mg/kg (P<0.001). The hypoglycaemic dose (50 mg/kg) was also determined by performing the oral glucose tolerance test in normal rabbits. The reduction in blood glucose level may be due to the reversible inhibition of α-glucosidases present in the brush-border of the small intestinal mucosa, higher rate of glycolysis as envisaged by the higher activity of glucokinase, as one of the key enzymes of glycolysis, and enhanced rate of glycogenesis as evidenced by the higher amount of liver glycogen present after oil administration. (Sepici A, Gurbuz I, Cevik C, Yesilada E. Hypoglycaemic effects of myrtle oil in normal and alloxan-diabetic rabbits. J. Ethnopharmacol. 2004; 93(2-3): 311-8.)

Oxidative stress is involved in the pathogenesis of numerous diseases. The polyphenol antioxidants in myrtle leaves have been studied. Antioxidant-rich fractions were prepared from myrtle (Myrtus communis L.) leaves liquid-liquid extraction (LLE) with different solvents. All myrtle extracts were very rich in polyphenols. In particular, hydroalcoholic extracts contain galloyl-glucosides, ellagitannins, galloyl-quinic acids and flavonol glycosides; ethylacetate extract and aqueous residues after LLE are enriched in flavonol glycosides and hydrolysable tannins (galloyl-glucosides, ellagitannins, galloyl-quinic acids), respectively. Qualitative and quantitative analysis for the single unidentified compound were also performed. Human LDL exposed to copper ions was used to evaluate the antioxidant activity of the myrtle extracts. Addition of these extracts did not affect the basal oxidation of LDL but dose-dependently decreased the oxidation induced by copper ions. Moreover, the myrtle extracts reduce the formation of conjugated dienes. The antioxidant effect of three myrtle extracts decreased in the following order: hydroalcoholic extracts, ethylacetate and aqueous residues after LLE. The extracts had the following IC₅₀: 0.36, 2.27 and 2.88 mM, when the sum of total phenolic compounds was considered after the correction of molecular weight based on pure compounds. Statistical analysis showed a significant difference among hydroalcoholic extracts vs. the ethylacetate and aqueous residues after LLE. These results suggest that the myrtle extracts have a potent antioxidant activity mainly due to the presence of galloyl derivatives. (Romani A, Coinu R, Carta S, Pinelli P, Galardi C, Vincieri F F, Franconi F. Evaluation of antioxidant effect of different extracts of Myrtus communis L. Free Radic Res. 2004 January; 38(1): 97-103).

Methanolic extracts of Myrtus communis seeds were reported inhibitory against S. aureus, Bacillus cereus and B. bronchiseptica (Bonjar G H. Antibacterial screening of plants used in Iranian folkloric medicine. Fitoterapia. 2004 March; 75(2):231-5).

The aqueous extracts of Myrtus communis L., have been reported to be antimutagenic (Hayder N, Kilani S, Abdelwahed A, Mahmoud A, Meftahi K, Ben Chibani J, Ghedira K, Chekir-Ghedira L. Antimutagenic activity of aqueous extracts and essential oil isolated from Myrtus communis L. Pharmazie. 2003 July; 58(7): 523-4.)

Extracts of Myrtus communis L., were found to be the most toxic against the mosquito Culex pipiens molestus (Diptera:Culicidae) (Traboulsi A F, Taoubi K, el-Haj S, Bessiere J M, Rammal S. Insecticidal properties of essential plant oils against the mosquito Culex pipiens molestus (Diptera:Culicidae). Pest Manag. Sci. 2002 May; 58(5): 491-5.)

The dimeric nonprenylated acylphloroglucinol semimyrtucommulone was obtained from the leaves of myrtle (Myrtus communis L.) as a 2:1 mixture of two rotamers. The known trimeric phloroglucinol myrtucommulone-A was also isolated and characterized spectroscopically as a silylated cyclized derivative. Myrtucommulone-A showed significant antibacterial activity against multidrug-resistant (MDR) clinically relevant bacteria, while semimyrtucommulone was less active. (Appendino G, Bianchi F, Minassi A, Sterner O, Ballero M, Gibbons S. Oligomeric acylphloroglucinols from myrtle (Myrtus communis). J. Nat. Prod. 2002 March; 65(3):334-8.)

Mild anti-inflammatory activity of Myrtus communis extracts is reported (Al-Hindawi M K, Al-Deen I H, Nabi M H, Ismail M A. Anti-inflammatory activity of some Iraqi plants using intact rats. J. Ethnopharmacol. 1989 September; 26(2):163-8.)

Antibacterial activity of Myrtus communis L., because of its acylphoroglucinols has been reported (Rotstein A, Lifshitz A, Kashman Y. Isolation and antibacterial activity of acylphloroglucinols from Myrtus communis L. Antimicrob. Agents Chemother. 1974 November; 6(5):539-42.)

Myrtle contains unique oligomeric non-prenylated acylphloroglucinols, whose antioxidant activity was investigated in various systems. Both semimyrtucommulone and myrtucommulone-A showed powerful antioxidant properties, protecting linoleic acid against free radical attack in simple in vitro systems, inhibiting its autoxidation and its FeCl₃- and EDTA-mediated oxidation. While both compounds lacked pro-oxidant activity, semimyrtucommulone was more powerful than myrtucommulone-A, and was further evaluated in rat liver homogenates for activity against lipid peroxidation induced by ferric-nitrilotriacetate, and in cell cultures for cytotoxicity and the inhibition of, TBH- or FeCl₃-induced oxidation. The results of these studies established semimyrtucommulone as a novel dietary antioxidant lead. (Rosa A, Deiana M, Casu V, Corona G, Appendino G, Bianchi F, Ballero M, Dessi M A. Antioxidant activity of oligomeric acylphloroglucinols from Myrtus communis L. Free Radic. Res. 2003 September; 37(9):1013-9).

Myrtucommulone (MC) and semimyrtucommulone (S-MC) are unique oligomeric, nonprenylated acylphloroglucinols contained in the leaves of myrtle (Myrtus communis L.). Although extracts of myrtle have been traditionally used in folk medicine for the treatment of various disorders, studies addressing select cellular or molecular pharmacological properties of these extracts or specific ingredients thereof are rare. MC and S-MC potently suppress the biosynthesis of eicosanoids by direct inhibiting cyclooxygenase-1 and 5-lipoxygenase in vitro and in vivo at IC₅₀ values in the range of 1.8 to 29 mM. Moreover, we show that MC and S-MC prevent the mobilization of Ca⁺² in polymorphonuclear leukocytes, mediated by G protein signaling pathways at IC₅₀ values of 0.55 and 4.5 mM, respectively, and suppress the formation of reactive oxygen species and the release of elastase at comparable concentrations. The isobutyrophenone core of MC as well as S-MC was much less potent or even not active at all. In addition, MC or S-MC only partially inhibited peroxide formation or failed to block Ca⁺² mobilization and elastase release when polymorphonuclear leukocytes were challenged with ionomycin that circumvents G protein signaling for cell activation. We conclude that, in view of their ability to suppress typical proinflammatory cellular responses, the unique acylphloroglucinols MC and S-MC from myrtle may possess an anti-inflammatory potential, suggesting their therapeutic use for the treatment of diseases related to inflammation and allergy. (Feisst C, Franke L, Appendino G, Werz O. Identification of molecular targets of the oligomeric nonprenylated acylphloroglucinols from Myrtus communis L., and their implication as anti-inflammatory compounds. J. Pharmacol. Exp. Ther. 2005, October; 315(1):389-96. Epub 2005 Jul. 13.)

Recently α-glucosidase inhibition activity of aqueous extract of Myrtle has been reported. (Onal S., Timur S Okutucu B., and Zihnioglu F., Prep. Biochem. Biotechnol., 2005, 35(1), 29-36.) α-Glucosidase inhibitors are used in the management of non-insulin-dependent diabetes mellitus (NIDDM). They act by reversible inhibition of the gastrointestinal sucrase, glucoamylase, dextrinase, maltase and isomaltase enzymes. These enzymes normally catatlyse the conversion of dietary starch and sucrose into absorbable monosaccharides. Enzyme inhibition therefore delays and reduces the peak of postprandial blood glucose. (McMorran J., Damian C. Crowther, McMorran S., Prince C., YoungMin S., and Pleat J., General Practice Note Book, DTB 1999, 3 [symbol] (11), 84-87.) They have been also used as inhibitors of tumor metastasis, antiobesity drugs, fungistatic compounds, insects antifeedants, antiviral and immune modulators. (El Ashry E. S. H., Rashed N., and Shobier A. H. S., Pharmazie, 2000, 55, 251-262). Agents with α-glucosidase inhibitory activity has been useful as oral hypoglycemic drugs for the control of hyperglycemia in patients with type 2; noninsulin-dependent, diabetes mellitus (NIDDM). Investigation of some medicinal herbs: Urtica dioica, Taraxacum officinale, Viscum album, and Myrtus communis with α-glucosidase inhibitor activity was conducted to identify a prophylactic effect for diabetes in vitro. All plants showed differing potent α-glucosidase inhibitory activity. However, Myrtus communis L., strongly inhibited the enzyme (IC₅₀=38 μg/mL). The inhibitory effect of these plants and some common antidiabetic drugs against the enzyme source (baker's yeast, rabbit liver, and small intestine) were also searched. Separation of the active material from Myrtus communis L., by HPLC shows that only one fraction acts as an α-glucosidase inhibitor. (Onal S, Timur S, Okutucu B, Zihnioglu F. Inhibition of α-glucosidase by aqueous extracts of some potent antidiabetic medicinal herbs. Prep Biochem Biotechnol 2005; 35(1):29-36). An ethanol-water extract of Myrtus communis L., (2 g/kg) administered intragastrically 30 min before streptozotocin abolished the initial hyperglycaemic without affecting the second phase. Myrtus extract given prior to streptozotocin and repeated at 24 h and 30 h, did not allow hyperglycaemia to develop until after 48 h. Administration of Myrtus extract 48 h after streptozotocin significantly reduced the hyperglycaemia and this effect was maintained by its repeated administration. Myrtus extract had no effect on the blood glucose level of normal mice. These studies confirm the “folk-medicine” indication of Myrtus extract as potentially useful in the treatment of diabetes mellitus. (Elfellah M S, Akhter M H, Khan M T. Anti-hyperglycaemic effect of an extract of Myrtus communis L., in streptozotocin-induced diabetes in mice. J. Ethnopharmacol. 1984 August; 11 (3):275-81.)

The U.S. Pat. Nos. 6,649,660 and 6,921,539 to Ninkov lists Myrtus communis L., as a source of natural carvacol and thymol for the treatment of infectious diseases using an intravenous preparation. The U.S. Pat. No. 6,844,369 to Ninkov reports an invention of a pesticidal compound wherein naturally derived thymol or Carvacol is obtained from Myrtus communis L.

The U.S. Pat. No. 6,818,234 to Nair et al., reports the use of food supplements that contain extract of Myrtus communis L., as a source of cyaniding 3-glucosides for pain relief and relief of inflammation because of the inhibitory properties mediated by cyclooxygenase and more particularly by cyclooxygenase-2.

The U.S. Pat. No. 6,284,289 to Van den Berghe describes an antiviral preparation containing extract of Myrtus communis L.

### DETAILS OF INVENTION

The aerial parts (8 kg dry wt) of Myrtus communis L. were collected from village Kabal of Swat district, NWFP, Pakistan, at an elevation of 1800 M in May-June 2003 and was identified by Mr. Mahboob-ur-Rahman (Assistant Professor), Department of Botany, Govt Jahanzeb Post Graduate College, Saidu Sharif, Swat, NWFP, Pakistan. A voucher specimen (CM-03) was deposited in the herbarium of the botany department. The freshly collected air-dried powdered plant material (8 Kg) was crushed and extracted by maceration in 80% methanol for 10 days (3×50 L). The combined methanol extract was evaporated and the concentrated viscous extract was partitioned between n-7-hexane, ethyl acetate and butanol. The ethyl acetate fraction (70 g) was fractionated by VLC over silica gel (1.4 kg), and eluted with hexane and gradients of chloroform up to 100% and methanol up to 20%. As a result of this, five sub-fractions were obtained. Sub fraction Fmc-3 (Compounds 4-7, 100 mg), on repeated silica gel column chromatography and elution with 20% ethyl acetate hexane, yielded usnic acid (compound 9), tectochrysine (compound 5), and betulin (compound 15). Similarly sub-fraction Fmc-5 on further purification by silica gel column chromatography and elution with 30% ethyl acetate-hexane yielded compounds, cearoin (compound 6), sideroxyline (compound 8), and oleanolic acid (compound 14). The hexane soluble fraction, obtained from crude methanolic extract, was subjected to column chromatography using 10% ethyl acetate-hexane as a mobile phase, yielded compounds, myrtucommulone-D, myrtucommulone-E, myrtucommulone-C, and mytocummolone-B. Similarly, the methanol soluble fraction was fractionated on a polyamide column, by using 30% acetone in chloroform as mobile phase to obtain erythrodiol (compound 13), ursolic acid (compound 10), corosolic acid (compound 11), arjunolic acid (compound 12), and β-sitosterol (compound 7).

Optical rotations were measured on a JASCO DIP 360 polarimeter. infrared spectra were recorded on a JASCO 302-A spectrophotometer. EI-MS and HREI-MS were recorded on Jeol JMS HX 110 with data system and on JMS-DA 500 mass spectrometers. The ¹H- and ¹³C-NMR spectra were recorded on Bruker NMR spectrometers, operating at 500 and 400 MHz (100 and 125 MHz for ¹³C). The chemical shifts values are reported in ppm (δ) units and the coupling constants (j) are given in Hz.

For thin layer chromatography (TLC), precoated aluminum sheets (silica gel G-60F-254, E. Merck) were used. Visualization of the TLC plates was achieved under UV at 254 and 366 nm and by spraying with cerric sulfate reagent. Solvent system n-hexane-ethyl:acetate (7:2, 9.5:0.5) was used. The structures of compounds 1, 2 and 4 were unambiguously deduced by single-crystal X-ray diffraction analysis. The known compounds mytocummolone-B (4) (Rotstein A., Lifshitz A., Kashman Y., Isr. Antimicrob. Agents Chemother. 1974, 6, 539-542; Kashman Y., Rotstein A., and Lifshitz A., Tetrahedron, 1974, 30, 991-997; Appendino G., Bianchi F., Minassi A., Sterner O., Ballero M., and Gibbons S., J. Nat. Prod., 2002, 65, 334-338). tectochrysine (compound 5) (Debral L. Taylor, Mohinder Kang, Tara M. Brenan, Gordon Bridges C., Prasad S. Sunkara, and Stanley Tyms, Antimicrobial Agents and Chemotherapy, 1994, 1780-1787), 2,5-dihydroxy-4-methoxybenzophenone (cearoin) (Compound 6) (Lounasmaa M., Puri H. S., and Widen C. J., Phytochemistry, 1977, 16, 1851)-sitosterol (compound 7) (Tharworn J., Vichai R., Pittaya T., and Thawatchai S., Phytochemistry, 1983, 22(2), 625-626) sideroxylin (De Souza Guimaraes I. S., Gotlieb O. T., Souza Andrade C. H., and Magalhaes M. T., Phytochemistry, 1975, 14, 1452-1453), usnic acid (compound 9) (Hillis W. E., and Koichiro Isoi, Phytochemistry, 1965, 4, 541-550), ursolic acid (compound 10) (Lounasmaa M., Widen C. J., and Reichstein T., Helv. Chem. Acta., 1971, 54, 2850), corosolic acid (compound 11) (Sakakibaraj., Kaiya T., Fukunda H., and Ohki T., Phytochemistry, 1983, 22, 2553), arjunolic acid (compound 12) (Furuya T., Orihara Y., and Hayashi C., Phytochemistry, 1987, 26, 715), erythrodiol (compound 13) (King F. E., King T. J., Ross J. M., J. Chem. Soc., 1954, 3995), oleanolic acid (compound 14) (Xue H.-Z., Lu Z.-Z., Konno C., Soejarto D. D., Cordell G. A., Fong H. H. S., and Hodgson W., Phytochemistry, 1988, 27, 233), and betulin (compound 15) (Fuchino H., Satoh T., and Tanaka N., Chem. Pharm. Bull., 1995, 43, 1937) were also obtained. The triterpenes (compounds 7, 10-15) and the flavonoids (compounds 5, 8) were isolated for the first time from the Myrtus communis L. The new compounds 1-3, as well as known compound 4, exhibited strong inhibition of α-glucosidase enzyme. Among all compounds myrtocummolone-C (Compound 3) was found to be the most potent α-glucosidase inhibitor with an IC₅₀=35.4±1.15 μM.

1. Myrtucommulone-D (Compound 1)

Yellowish Crystals (27 mg); Melting Point 138-140° C.; [α]³⁰_{D} +375.00 (c=0.8, CHCl₃); infrared νₘₐₓ cm⁻¹ (CHCl₃): 3450 (OH), 2968 (aromatic CH), 1719 (saturated ketone), 1601 (aryl), 1659 (C═C), 1590 (enolic-1,3-diketone system), 1250-1383 (C—C); ¹H, ¹³C-NMR δ (see Table-1); CIMS: m/z 651 [M+1]⁺ (FIG. 1)

Myrtucommulone-D (Compound 1) was isolated from the methanolic extract of Myrtus communis L., as yellow crystals. The compound 1 was assigned the formula C₃₈H₅₀O₉ on the bases ion peak at m/z 651 [M+1]⁺ in CIMS and X-ray and NMR spectral data. The infrared spectrum of compound 1 showed absorption bands at 3500 (OH), 2968 (aromatic CH), 1710 (saturated ketone), 1620 (aryl), and 1580 (enolic 1,3-diketone system).

The ¹H-NMR spectrum showed the presence of two isopropyl, one isobutylidene group and eight methyls bound to non-protonated aliphatic carbons. Six doublets resonated at δ 0.66 [symbol] =6.8 Hz), 0.88 [symbol] =6.8 Hz), 0.75 [symbol] =6.9 Hz), 0.89 [symbol] =7.0 Hz), 1.16 [symbol] =6.6 Hz), and 1.20 [symbol] =7.0 Hz) were assigned to C-9, C-9″, C-10″, C-10, C-10′, and C-9′ methyl protons, respectively. The singlets, resonating at δ 1.29, 1.32, 1.40, 1.56, 1.57, 1.62, 1.39, and 1.47, were due to C-12′, C-12″, C-14′, C-13′, C-1′, C-14″, C-13″, and C-11″ methyl protons, respectively. The two multiplets at δ 2.01 and 2.35, and a septet at δ 3.89, were due to C-8, C-8″ and C-8′ methine protons, respectively. A downfield double doublet at δ 4.18 [symbol] _{7,8}, =3.6, Hz J_{7,1}=3.5 Hz) was assigned to C-7 methine proton. A septet for C-8′ at δ 3.89, and a doublet for two methyl groups at δ 1.20 was characteristic of a (CH₃)₂CHCO— group in this class of compounds. The loss of 43 a.m.u. from M⁺ further supported the presence of a (CH₃)₂CHCO— group (Winter A. G., Willeke L., Naturewissenschaften, 1951, 38, 262-264; Hayder N., Abdelwahed A., Kilani S., Ammar R. B., Mahmoud A., Ghedira K., and Chekir-Ghedira L., Mutat. Res., 2004, 564(1), 89-95).

TABLE 1
¹H and ¹³C NMR data of (Compound 1) and (Compound 2) in (CDCl₃, 400 MHz
for ¹H and 100 MHz for ¹³C)

| | Myrtucommulone- | Myrtucommulone- | Myrtucommulone- | Myrtucommulone- |
|---|---|---|---|---|
| C. | D (Compound 1) | D (Compound 1) | E (Compound 2) | E (Compound 2) |
| No | ¹³C (δ) | ¹H NMR, δ [symbol] Hz) | ¹³C (δ) | ¹H NMR, δ [symbol] Hz) |
| 1 | 106.7 (C) | — | 103.2 (C) | — |
| 2 | 150.6 (C) | | 151.0 (C) | |
| 3 | 108.5 (C) | | 106.1 (C) | |
| 4 | 161.4 (C) | | 160.9 (C) | |
| 5 | 108.6 (C) | | 110.3 (C) | |
| 6 | 153.5 (C) | | 153.3 (C) | |
| 7 | 28.9 (CH) | 4.18, dd, 3.6, 3.5 | 32.2 (CH) | 4.38, d, 3.6 |
| 8 | 32.2 (CH) | 2.35 m | 35.4 (CH) | 1.98 m |
| 9 | 16.0 (CH₃) | 0.66, d, 6.8 | 18.5 (CH₃) | 0.81, d, 3.6 |
| 10 | 20.2 (CH₃) | 0.89, d, 7.0 | 19.4 (CH₃) | 0.94, d, 6.9 |
| 1′ | 45.7 (CH) | 3.67, d, 5.9 | 111.9 (C) | |
| 2′ | 204.9 (C) | | 197.6 (C) | |
| 3′ | 56.2 (C) | | 56.2 (C) | |
| 4′ | 211.9 (C) | | 211.5 (C) | |
| 5′ | 54.9 (C) | | 47.2 (C) | |
| 6′ | 100.1 (C) | | 167.2 (C) | |
| 7′ | 210.2 (C) | | 209.5 (C) | |
| 8′ | 39.9 (CH) | 3.89, m | 40.2 (CH) | 3.83, m |
| 9′ | 20.4 (CH₃) | 1.20, d, 7.0 | 19.7 (CH₃) | 0.83, d, 7.1 |
| 10′ | 17.9 (CH₃) | 1.16, d, 6.6 | 17.7 (CH₃) | 1.26, d, 6.0 |
| 11′ | 25.4 (CH₃) | 1.57, s | 23.8 (CH₃) | 1.57, s |
| 12′ | 24.2 (CH₃) | 1.29, s | 25.2 (CH₃) | 1.29, s |
| 13′ | 24.7 (CH₃) | 1.56, s | 25.5 (CH₃) | 1.56, s |
| 14′ | 19.2 (CH₃) | 1.40, s | 24.9 (CH₃) | 1.40, s |
| 1″ | 111.6 (C) | | 110.3 (C) | |
| 2″ | 197.5 (C) | | 197.4 (C) | |
| 3″ | 57.3 (C) | | 56.1 (C) | |
| 4″ | 213.8 (C) | | 211.5 (C) | |
| 5″ | 47.4 (C) | | 47.5 (C) | |
| 6″ | 167.4 (C) | | 167.2 (C) | |
| 7″ | 32.0 (CH) | 4.37, d, 3.2 | 31.9 (CH) | 4.40, d, 3.4 |
| 8″ | 34.2 (CH) | 2.01, m | 34.3 (CH) | 1.95, m |
| 9″ | 20.0 (CH₃) | 0.88, d, | 20.7 (CH₃) | 1.26, d, 6.0 |
| 10″ | 18.3 (CH₃) | 0.75, d, | 18.5 (CH₃) | 0.79, d, |
| 11″ | 24.9 (CH₃) | 1.47, s | 24.9 (CH₃) | 1.41, s |
| 12″ | 22.1 (CH₃) | 1.32, s | 23.9 (CH₃) | 1.32, s |
| 13″ | 25.3 (CH₃) | 1.39, s | 25.1 (CH₃) | 1.77, s |
| 14″ | 24.3 (CH₃) | 1.62, s | 25.0 (CH₃) | 1.64, s |

*The ¹H-¹³C connectivity and ¹³C multiplicities were deduced according to HMQC and DEPT experiments.

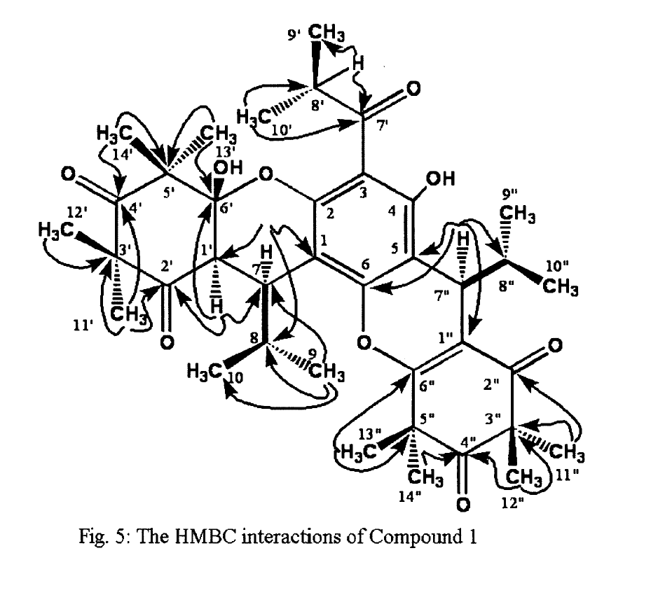

The ¹³C-NMR spectrum (BB, DEPT) (Table-1) showed thirty eight signals, including fourteen methyls, six methines, and eighteen quaternary carbons. In the HMBC spectrum (FIG. 5), the C-1′ methine proton (δ 3.67) showed correlations with C-2′ (δ 204.9), C-6′ (δ 100.1), and C-7 (δ 28.9).

### FIG. 5: The HMBC Interactions of Compound 1

The C-7 methine proton showed correlations with C-1 (δ 106.7), C-1′ (δ 45.7), C-8 (δ 32.2), C-2′ (δ 204.9), C-2 (δ 150.6) and C-6′ (δ 100.1). The C-9′ methyl (δ 1.20), and C-8′ methine (δ 3.89) protons showed correlations with the exocyclic carbonyl carbon (C-7′) (δ 210.2). Similarly C-11′ (δ 1.35) and C-12′ (δ 1.29) methyl protons exhibited interactions with C-2′ (δ 204.9), and C-4′ (δ 211.9). Furthermore, C-13′ (δ 1.56) and C-14′ (δ 1.40) methyl protons showed HMBC interactions with C-4′ (δ 211.9), C-6′ (δ 100.1), and C-5′ (δ 54.9).

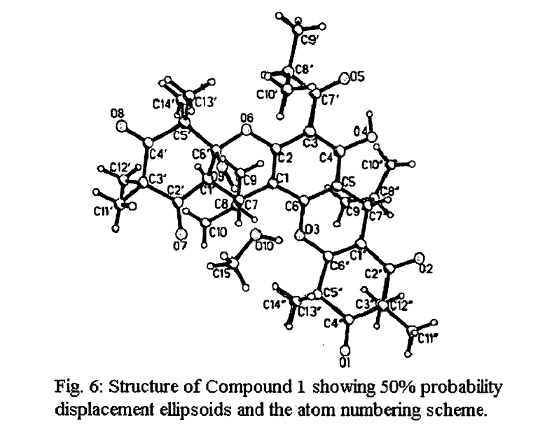

Finally the structure and relative stereochemistry of myrtucommulone-D (Compound 1) was unambiguously deduced by a single-crystal X-ray diffraction analysis (FIG. 6).

### FIG. 6. Structure of Compound 1 Showing 50% Probability Displacement Ellipsoids and the Atom Numbering Scheme.

Compound 1 was obtained as a colorless block crystals (0.97×0.56×0.53 mm) and its X-ray analysis showed normal bond lengths and bond angles (Allen, F. H., Kennard, O., Watson, D. G., Brammer, L., Orpen, A. G., and Taylor, R. J. Chem. Soc. Perkin Trans. 2, 1987, pp. S1-S19.) The pentacyclic benzopryanoxanthene of 1 is nearly a planar molecule in which rings A and E slightly deviate from the planes of rings B, C and D. Both isopropyl groups at C-7 and C-7″ were oriented in the same direction, but opposite to the direction of C-6′ hydroxyl group. The rings A and B are transfused with each other and hydroxyl group at C-6′ is twisted towards the plane of C-2′ carbon, the C-2′-C-1′-C6′-O9 torsion angle being 50.78 (17)⁰.

X-Ray Data of Myrtucommulone-D (Compound 1)

A slab shaped yellow crystals of compound 1, with dimension 0.97×0.56×0.53; mm was selected for X-ray diffraction studies. C₃₈H₅₀O₉,: Mol. Wt. 650.7984; monoclinic; a=19.1180 (9), b=10.2680 (5), and, c=20.1967 (9), A⁰, V=3834.3(3), A⁰³, space group=P2 (1)/n, Z=4, D_{calc.}=1.183, mg/m³, F (000)=1472, Mo-Kα (λ0.7107⁰A). Intensity data of compound 1 was collected on a Siemens Smart CCD 1-K area-detector diffractometer. (Seimens. SMART and SAINT. Siemens Analytical X-ray instruments Inc., Madison, Wis., USA, 1996). Data reductions were performed using SAINT. The structure was solved by direct methods (Altomare A., Cascarano M., Giacovazzo C., Guagliardi A., J. Appl. Cryst. 1993, 26, 343) and refined by full-matrix least squares on F² using the SHELXTL-PC package. (Sheldrick G. M., SHELXTL-PC (Version 5.1), Siemens Analytical Instruments, Inc., Madison, Wis., 1997). The intensity data within the θ range 2.09-25.00, were collected at 293(2) K. A total of 18816 reflections were recorded, of which 6743 reflections were observed on the basis of I>2 s (Compound 1). The final R(1) and Rw(1) were 0.0479, and 0.1365, respectively. The figure was plotted with the aid of ORTEP. (Johnson C. K., ORTEPII′, Report ORNL-5138. Oak Ridge National Laboratory, Tennessee, USA, 1976).

2. Myrtucommulone-E (Compound 2):

Yellowish Crystals (18 mg), Melting Point 163-165° C.; [α]³⁰_{D} −166.7° (c=1.2, CHCl₃) infrared νₘₐₓ cm⁻¹ (CHCl₃): 3422 (OH), 2968 (aromatic CH), 1719 (saturated ketone), 1601 (aryl), 1659 (C═C), 1590 (enolic-1,3-diketone system), 1250-1383 (C—C); ¹H, ¹³C-NMR δ (see Table-1); HREI-MS: m/z 632.7834, C₃₈H₄₈O₈ (FIG. 2).

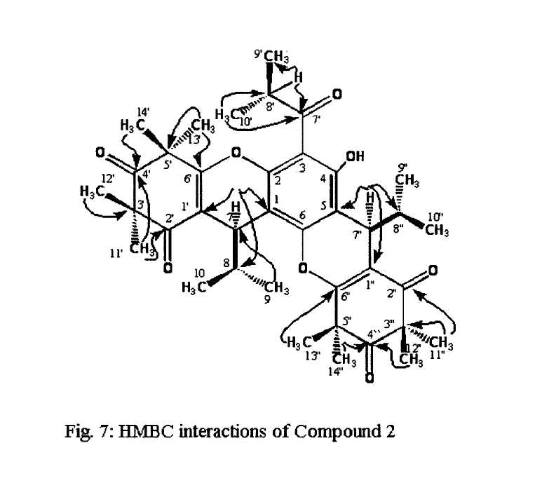

Myrtucommulone-E (Compound 2) was isolated from the methanolic extract of Myrtus communis L., as yellow crystals. The compound 2 had molecular formula C₃₈H₄₈O₈, as derived from the ion peak at m/z 633 [M+1]⁺ in Cl-MS and X-ray and NMR spectral data The infrared spectrum of 2 showed absorption bands at 3430 (OH), 2968 (aromatic CH), 1719 (saturated ketone), 1617 (aryl), and 1580 (enolic 1,3-diketone system). The NMR spectral data of compound 2 showed the distinct resemblance with that of compound 1 except for the absence of C-1′ methine proton in ¹H-NMR and the presence of additional olefinic signals at δ 111.9 and 167.2 in ¹³C-NMR spectrum (Table-2) of compound 2, indicating the presence of a double bond between C-1′ and C-6′ in compound 1. The position of olefinic bond at C-1′ and C-6′ was further confirmed on the basis of HMBC spectrum in which the C-7 methine proton (δ 4.38) showed correlations with C-1 (δ 103.2), C-1′ (δ 11.9), C-8 (δ35.4), and C-2′ (δ 197.5) (FIG. 7)

### FIG. 7. HMBC Interactions of Compound 2

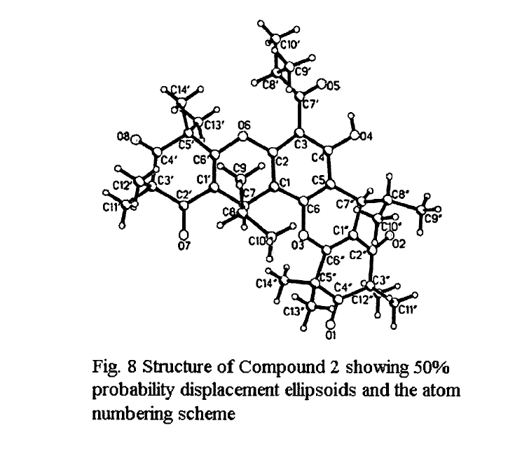

The structure of the compound 2 was also unambiguously deduced by the single-crystal X-ray diffraction analysis (FIG. 8).

### FIG. 8. Structure of Compound 2 Showing 50% Probability Displacement Ellipsoids and the Atom-Numbering Scheme.

The compound 2 was obtained as a colorless plate crystals (0.76×0.25×0.18 mm) and its X-ray analysis showed the normal bond lengths and bond angles. Allen, F. H., Kennard, O., Watson, D. G., Brammer, L., Orpen, A. G., and Taylor, R. J. Chem. Soc. Perkin Trans. 2, 1987, pp. S1-S19.) The pentacyclic benzopryanoxanthene 2 was found to be nearly a planar moiety in which ring A and E were slightly deviate from the planes of ring B, C and D. Both isopropyl groups at C7 and C7″ are oriented in the same direction.

X-Rays Data of Myrtucommulone-E (Compound 2)

A plate shaped colorless crystal of compound 2 with dimension 0.76×0.25×0.18 mm was selected for X-ray diffraction studies. C₃₈H₄₈O₈: Mr 632.76; monoclinic; a=13.317 (2), b=15.201(2), c=18.692(3) A⁰, V=3596.3(9) A⁰³, space group=P2(1)/c, Z=4, D_{calc.}=1.169 g/cm³, F(000)=1360.0, Mo-Kα (λ 0.7107⁰A). Intensity data of compound 2 was collected on a Siemens Smart CCD 1-K area-detector diffractometer. (Siemens. SMART and SAINT. Siemens Analytical X-ray instruments Inc., Madison, Wis., USA, 1996.) Data reductions were performed using SAINT. The structure was solved by direct methods Altomare A., Cascarano M., Giacovazzo C., Guagliardi A., J. Appl. Cryst. 1993, 26, 343) and refined by full-matrix least squares on F² using the SHELXTL-PC package. (Sheldrick G. M., SHELXTL-PC (Version 5.1), Siemens Analytical Instruments, Inc., Madison, Wis., 1997) The intensity data within the θ range 1.61-25.00 were collected at 293 (2) K. A total of 17852 reflections were recorded, of which 6320 reflections were observed on the basis of I>2 s (1). The final R and Rw were 0.0566 and 0.1563, respectively. The figure-4 was plotted with the aid of ORTEP. (Johnson C. K., ORTEPII', Report ORNL-5138. Oak Ridge National Laboratory, Tennessee, USA, 1976)

Crystallographic data for compounds 1 and 2 has been deposited to Cambridge Crystallographic Data Center, 12 Union Road, Cambridge, CB/EZ, UK (Fax: 44-1223-336-033, e-mail: deposit@ccdc.cam.ac.uk).

3. Myrtucommulone-C (Compound 3):

Yellow amorphous powder (15 mg); [α]³⁰_{D} +13.0° (c=1.5, CHCl₃); infrared νₘₐₓ cm⁻¹ (CHCl₃): 3500 (OH), 2968 (aromatic CH), 1717 (saturated ketone), 1628 (aryl) and 1590 (enolic-1,3-diketone system). ¹H-, ¹³C-NMR δ (see Table-2). FABMS: m/z 651.0 [M+1]⁺

Myrtucommulone-C (Compound 3) was also isolated from the methanolic extract of Myrtus communis L., as an amorphous powder. The compound 3 was assigned the formula C₃₈H₅₀O₉ on the bases of an ion peak at m/z 651 [M+1]⁺ in FABMS and based on NMR data. The EI-MS of compound 3 exhibited a base peak at m/z 589 which resulted from the loss of H₂O and —C₃H₇ groups from M⁺. The infrared spectrum showed absorption bands (OH), 2968 (aromatic CH), 1710 (saturated —C═O), 1620 (aryl), and 1580 (enolic 1,3-diketone system). The NMR spectral data of compound 3 indicated its resemblance with the compound 2 except for the absence of C-6 and C-2″ ether linkage. The ¹³C-NMR spectrum (BB, DEPT) (Table-2) showed thirty eight signals, including fourteen methyls, five methines and nineteen quaternary carbons.

TABLE 2
¹H and ¹³C NMR data of (Compound 3) in (CDCl₃, 400 MHz for ¹H
and 100 MHz for ¹³C)

| C. | | ¹H, δ [symbol] |
|---|---|---|
| No. | ¹³C (δ) | Hz) |
| 1 | 111.9 (C) | — |
| 2 | 153.2 (C) | |
| 3 | 106.2 (C) | |
| 4 | 160.9 (C) | |
| 5 | 110.3 (C) | |
| 6 | 151.0 (C) | |
| 7 | 32.2 (CH) | 4.35, d, |
| | | 3.4 |
| 8 | 34.3 (CH) | 1.9 m |
| 9 | 18.4 (CH₃) | 0.79, br s |
| 10 | 19.4 (CH₃) | 0.79, br s |
| 1′ | 111.9 (C) | |
| 2′ | 197.4 (C) | |
| 3′ | 56.7 (C) | |
| 4′ | 211.5 (C) | |
| 5′ | 47.1 (C) | |
| 6′ | 167.2 (C) | |
| 7′ | 209.5 (C) | |
| 8′ | 40.2 (CH) | 3.9, m |
| 9′ | 20.7 (CH₃) | 1.24, d, |
| | | 6.0 |
| 10′ | 17.7 (CH₃) | 1.16, d, |
| | | 6.0 |
| 11′ | 23.8 (CH₃) | 1.35, s |
| 12′ | 25.2 (CH₃) | 1.41, s |
| 13′ | 31.8 (CH₃) | 1.60, s |
| 14′ | 24.9 (CH₃) | 1.40, s |
| 1″ | 111.7 (C) | |
| 2″ | 167.6 (C) | |
| 3″ | 47.5 (C) | |
| 4″ | 211.8 (C) | |
| 5″ | 56.2 (C) | |
| 6″ | 197.6 (C) | |
| 7″ | 31.9 (CH) | 4.38, d, |
| | | 3.4 |
| 8″ | 35.2 (CH) | 2.01, m |
| 9″ | 19.7 (CH₃) | 0.88, d, |
| | | 6.1 |
| 10″ | 18.5 (CH₃) | 0.75, d, |
| 11″ | 25.3 (CH₃) | 1.47, s |
| 12″ | 25.1 (CH₃) | 1.32, s |
| 13″ | 24.0 (CH₃) | 1.39, s |
| 14″ | 25.4 (CH₃) | 1.62, s |

Note:
The ¹H-¹³C connectivities and ¹³C multiplicities were deduced according to HMQC and DEPT experiments.

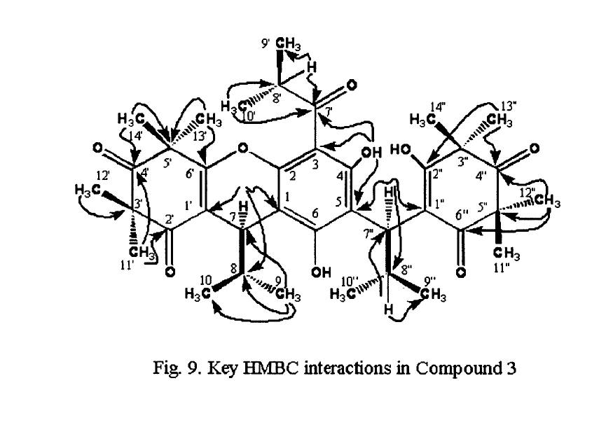

In HMBC spectrum (FIG. 9), the C-7 methine proton (δ 4.35) showed correlations with C-1 (δ 103.0), C-1′ (δ 111.9), C-8 (δ34.3), and C-2′ (δ 197.4). The presence of OH group at C-4 (δ 160.9) was further confirmed by the HMBC correlations of hydroxyl proton (δ 12.63) with exocyclic carbonyl C-7′ (δ 209.5), C-3 (δ 106.2), C-2 (δ 153.2), C-4 (δ 160.9), and C-5 (δ 110.3).

### FIG. 9. Key HMBC Interactions in Compound 3

4: Myrtucommolone-B (Compound 4)

Yellowish Crystals (10 mg); [α]³⁰_{D} 78.3° (c=1.2, CHCl₃); IR υₘₐₓ cm⁻¹ (CHCl₃) 3500 (OH), 2968 (aromatic CH), 1717 (saturated ketone), 1628 (aryl) and 1590 (enolic-1,3-diketone system) 3492 (OH), 1083 (simple ether bonds); HREI-MS: m/z 414.491 C₂₄H₃₂O₇ (calcd 414.487). (FIG. 4)

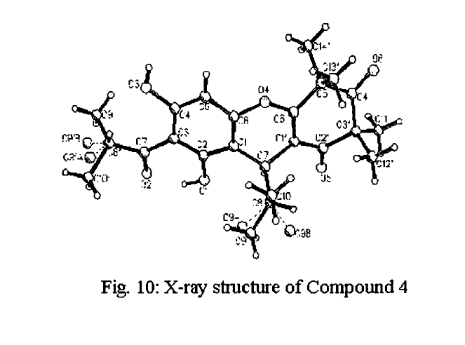

The structure and relative stereochemistry of myrtucommulone-B (Compound 4) was unambiguously deduced by a single-crystal X-ray diffraction analysis (FIG. 10). The structure was solved by direct method using the program SHELXS-97. Refinement of the structure by the method of least squares was done by SHELXL-97 program in the full-matrix isotropic-anisotropic approximation (over F²). The isopropyl fragments in C8 and C8′ are disordered.

### FIG. 10. X-Ray Structure of Compound 4

The X-Ray experiment was conducted on a STOE Stadi-4, by using MoK_{α} radiation (graphite monochromator). Crystals of 4 were grown an 15% ethylacetate in Hexane solution. General crystallographic data and conditions of X-Ray experiment given in Table 3.

TABLE 3
General crystallographic data and conditions of X-Ray experiment of
myrtucommulone-B (Compound 4)

| | |
|---|---|
| Molecular formula | C₂₄H₃₂O₇ |
| Molecular weight | 414.491 |
| Temperature, K° | 293 |
| Space group | P2₁/c, Z = 4 |
| a, Å | 9.123(2) |
| b, Å | 27.123(5) |
| c, Å | 9.329(2) |
| B | 99.19(3) |
| V, Å³ | 2278.8(8) |
| P, r/cm³ | 1.202 |
| Absorption coefficient, | 0.086 |
| (Mo)mm⁻¹ | |
| Size of crystal, mm | 0.00 × 0.00 × 0.00 |
| Range of angles, degrees | From 1.50 to 24.99 |
| Common number of | 4011 |
| reflections | |
| Number of reflections [I > 2σ(I)] | 2233 |
| R-factor [I > 2σ(I)] | R1 = 0.0795, wR2 = 0.1563 |
| R-factor (for all array) | R1 = 0.1294, wR2 = 0.1668 |
| Goodness-of-fit on F² | 1.147 |
| Largest diff. peak and hole | 0.172 and −0.175 eA⁻³ |

Antibacterial activity: Compounds 1 and 2 showed significant antibacterial activity against Staphylococcus aureus. The compound 9 showed significant activities against Salmonella typhi and Pseudomonas aeruginosa, while the compounds 7, 11, 12, 13, 14, and 15 showed significant activity against Salmonella typhi and Pseudomonas aeruginosa (Table-4).

α-Glucosidase Inhibition Studies: The phloroglucinol-type compounds 1-4 were found to be potent inhibitors of the α-glucosidase enzyme. The compounds 1-4 showed the inhibitory activity in a dose dependent manner. All compounds were more potent than the clinically used standard inhibitors e.g. deoxynojirimycin and acarbose, which are used in the type II diabetes. The compound 3 exhibited the highest activity among all phloroglucinols tested (Table-5).

Antibacterial Activity

All of the isolated compounds were screened against strains of Escherichia coli, Bacillus subtilis, Shigella flexneri, Staphylococcus aureus, Pseudomonas aeruginosa, and Salmonella typhi. For antibacterial screening, 3 mg of sample was taken and dissolved in 3 ml of DMSO. Molten nutrient agar (45 mL) was poured on sterile petri plates, where it was allowed to solidify. Bacterial lawn were made on these nutrient agar plates by dispensing 7 mL of sterile soft agar containing 100 μL of test-organism culture. Wells were duged with the help of a 6-mm sterile metallic borer at appropriate distance. Then, 100 μL of sample was poured into each well, and the plates were incubated at 37° C. for 24 h. The results, in terms of inhibition zone, were noted. The drug Imipenem, a broad-spectrum β-lactam antibiotic, was used as a positive control. As a negative control, DMSO was used. The results of these experiments are summarized in Table-4.

TABLE 4
Antibacterial Activities of Compounds 1, 2, 4, 7, 11-15. Relative to the
Standard Drug Imipenem. Inhibition zones are given in mm.

| Compound | EC | BS | SF | SA | PA | ST |
|---|---|---|---|---|---|---|
| Imipenem | 30 | 33 | 27 | 33 | 24 | 25 |
| 1 | — | 15 | 12 | 24 | — | 15 |
| 2 | 10 | 15 | 10 | — | 13 | 17 |
| 7 | — | 12 | — | 15 | 16 | 18 |
| 9 | 11 | 16 | 15 | 15 | 17 | 17 |
| 11 | — | 15 | 15 | — | 17 | 17 |
| 12 | 9 | 15 | 13 | 15 | 12 | 16 |
| 13 | 16 | 17 | — | 11 | 12 | 19 |
| 14 | — | 15 | 10 | — | 16 | 16 |
| 15 | — | — | 15 | — | 18 | 16 |

Abbreviations:
EC, Escherichia coli;
BS, Bacillus subtilis;
SF, Shigella flexneri;
SA, Staphylococcus aureus;
PA, Pseudomonas aeruginosa;
ST, Salmonella typhi.

Enzyme Inhibition Assay:

α-Glucosidase (E.C.3.2.1.20) enzyme inhibition assay has been performed according to the slightly modified method of Matsui et al., (Matsui T., Yoshimoto C., Osajima K., Oki T., and Osajima Y., Biosci. Biotech. Biochem., 1996, 60, 2019-2022). α-Glucosidase (E.C.3.2.1.20) from Saccharomyces species, purchased commercially (Wako 076-02841; Wako Pure Chemical Industries Ltd., 1-2 Doshomachi 3-Chome, Chuo-ku, Osaka 540-8605, Japan). The enzyme inhibition was measured spectrophotometrically at pH 6.9 and at 37° C. using 0.7 mM p-nitrophenyl α-D glucopyranoside (PNP-G) as a substrate and 500 m units/mL enzyme, in 50 mM sodium phosphate buffer containing 100 mM NaCl. 1-Deoxynojirimycin (0.425 mM) and acarbose (0.78 mM) were used as positive control. The increment in absorption at 400 nm, due to the hydrolysis of PNP-G by α-glucosidase, was monitored continuously on microplate spectrophotometer (Spectra Max Molecular Devices, USA).

TABLE 5
The α-glucosidase inhibitory activity

| No. of | | |
|---|---|---|
| Compound | Name | IC₅₀ μM |
| 1 | Myrtucommulone-D | 84.3 ± 3 |
| 2 | Myrtucommulone-E | 46.6 ± 0 |
| 3 | Myrtucommulone-C | 35.4 ± 1.15 |
| 4 | Myrtucommulone-B | 39.99 ± 1.00 |
| Standard | Deoxynojirimycin | 425.6 ± 8.14 |
| | Acarbose | 780 ± .028 |

What is claimed is:

1. A compound selected from a group of acylphloroglucinols consisting of myrtucommulone-D (compound 1) with molecular formula of C₃₈H₅₀O₉ and molecular weight of 650 and chemical structure of FIG. 1,
FIG. 1
myrtucommulone-E with molecular formula of C₃₈H₄₈O₈ and molecular weight of calculated 632.78 and chemical structure of FIG. 2,
FIG. 2,
and myrtucommulone-C (Compound 3) with molecular formula of C₃₈H₅₀O₉ and molecular weight of 650 and chemical structure of FIG. 3,
FIG. 3
and their enantiomers, diastereomers, or a pharmaceutically-acceptable salt, hydrate, solvate, or prodrug thereof.

2. As claimed in claim 1, wherein the said acylphloroglucinols are derived from a methanolic extract of the aerial parts of an evergreen shrub, Myrtus communis L., of Myrtaceae family commonly known as Myrtle (English).

3. As claimed in claim 1, wherein the said acylphloroglucinols are inhibitors of α-glucosidase enzyme.

4. As claimed in claim 1, wherein the said acylphloroglucinols are inhibitors of bacteria.
